(19) United States
(12) Patent Application Publication
(10) Pub. No.: US 2026/0106007 A1
(43) Pub. Date: Apr. 16, 2026

(54) INFORMATION PROCESSING SYSTEM, INFORMATION PROCESSING TERMINAL, HEALTH MANAGEMENT FUNCTION PROVIDING METHOD, AND RECORDING MEDIUM

(71) Applicant: OMRON HEALTHCARE Co., Ltd., Kyoto (JP)

(72) Inventors: Shingo YAMASHITA, Kyoto (JP); Miki OKUGAWA, Kyoto (JP); Yuka SUZUKI, Kyoto (JP)

(21) Appl. No.: 19/389,868

(22) Filed: Nov. 14, 2025

Related U.S. Application Data
(63) Continuation of application No. PCT/JP2024/002950, filed on Jan. 30, 2024.

(30) Foreign Application Priority Data
Jul. 6, 2023 (JP) 2023-111120

(51) Int. Cl. G16H 20/00; G16H 40/67

(52) U.S. Cl. CPC G16H 20/00; G16H 40/67

(57) ABSTRACT

An information processing system at least including an information processing terminal configured to execute an application recording medium for providing a health management function relating to management of health of a user including a user information acquisition unit configured to acquire user information including an indicator relating to health of the user; a user suitability determination unit configured to perform a determination of suitability of the user for the health management function to be provided, on a basis of an indicator included in the user information; and a setting unit configured to set a mode of providing the health management function according to a result of the determination.

### CROSS-REFERENCE TO RELATED APPLICATIONS

This application is the U.S. national stage application filed pursuant to 35 U.S.C. §§ 365(c) and 120 as a continuation of international patent application No. PCT/JP2024/002950, filed Jan. 30, 2024, which application claims priority to Japanese patent application No. 2023-111120, filed Jul. 6, 2023, which applications are incorporated herein by reference in their entireties.

### FIELD

The present invention belongs to a technical field related to healthcare and relates to an information processing system, an information processing terminal, a health management function providing method, and a recording medium (or “recording medium”).

### BACKGROUND

In recent years, interest in health care performed by individuals has increased, and a system capable of providing a health management function including presenting information such as behavioral changes leading to treatment of a disease to a patient has been proposed (for example, Patent Document 1: JP 2019-24943 A).

Patent Document 1 discloses a system including a server and a user terminal in which, when a patient provides medical information such as their vitals data by the user terminal (for example, a smartphone or the like), information for treatment of a disease in accordance with the individual patient is presented via the user terminal on the basis of the medical information.

According to such a system, for a disease (for example, a lifestyle-related disease) in which the content of daily activities of a patient affects treatment, information for prompting an appropriate action is provided to the patient so that the behavior of the patient can be corrected, leading to treatment of the disease.

### SUMMARY

### Technical Problem

However, in the case of using the system as described above, for example, when there is a problem in the authenticity of information provided from the patient via the user terminal or in the understanding of the patient of information related to the treatment of the disease presented to the patient by the system due to a decline in the cognitive function of the patient, there is a possibility that this may lead to wrong treatment behavior. In addition, depending on the physical condition of the patient, the content of the function provided from the system to the patient may be inappropriate (for example, information similar to that of when in a healthy state may be presented to a patient with a poor physical condition).

In view of the above-described problems, an object of the present invention is to provide a function in an appropriate manner in accordance with the characteristics or the state of a user when providing the function related to user health management to the user.

### Solution to Problem

The present invention employs the following configurations to solve the above-described problems. That is, an information processing system at least including an information processing terminal configured to execute an application recording medium (or recording medium) for providing a health management function relating to management of health of a user includes: a user information acquisition unit configured to acquire user information including an indicator relating to health of the user; a user suitability determination unit configured to perform a determination of suitability of the user for the health management function to be provided, on a basis of an indicator included in the user information; and a setting unit configured to set a mode of providing the health management function according to a result of the determination.

Here, “health management” refers not only to so-called preventive medical care for healthy persons but also to medical practice for patients with diseases and injuries. The “health management function” also includes transmission of vitals data of the user to a medical professional such as a physician. The “user information” includes various information and includes biological information such as blood pressure, pulse, body temperature, weight, and electrocardiogram; body function information such as eyesight, hearing, muscle strength, and cognitive function; attribute information such as age and gender; and symptoms (for example, pain, numbness, and the like) in the case of a disease or injury and the site, intensity, and the like thereof. The term “acquiring” mentioned here includes receiving a manual input from the user, receiving sensing information by a sensor device, receiving a measurement result by a biological information measurement device, and the like. The sensor device and the measurement device may be integrally formed with the information processing terminal. The “indicator” is not limited to one and may be plural depending on the type of information to be acquired. Further, “setting the mode of providing the health management function”includes adding a restriction to the function to be provided.

With such a configuration, it is possible to provide a health management function optimized for the user at the time of use after determining the suitability of the user for the function provided by the application based on the user information. Specifically, for example, in a case where a decline in cognitive function is suspected, the provision of the health management function may be restricted, and the function can be provided only to a user who can correctly utilize the health management function.

The indicator may include an indicator relating to a cognitive function of the user, and setting a mode of providing the health management function performed by the setting unit may include presenting, when providing the health management function, information encouraging early examination by a physician. Also, in a case where a mode of providing the health management function is set by the setting unit to present information encouraging early examination by a physician, and information indicating that an examination by a physician has been made is not input within a predetermined time period, the setting unit may further set a mode of providing a health management function to stop provision of the health management function.

According to this, it is possible to prevent the health management function from being continuously provided in a state where there is a doubt relating to a cognitive function, and it is possible to reduce a possibility that an inappropriate course of action is proposed based on an input of erroneous information or a wrong treatment action is performed due to a failure to correctly understand medical practice information provided from the system.

Also, the information processing terminal may include a communication unit, the health management function may include transmitting the user information via the communication unit, and the setting unit setting a mode of providing the health management function may include setting to add the result of the determination for the suitability to the user information and transmit the user information.

For example, in a case where the user transmits vitals data such as blood pressure, when the cognitive function of the user has decreased, there may be doubt about the accuracy of the transmitted vitals data. When a physician examines a treatment strategy or the like of the user based on the transmitted vitals data, it is assumed that the vitals data is accurate. Thus, by adding the result of the determination on the cognitive function to the vitals data, the physician can perform examination based on the degree of accuracy of the data.

Also, the information processing terminal may include an image display unit, the health management function may include presenting information via the image display unit, the indicator may include an indicator relating to visual acuity of the user, and the setting unit setting a mode of providing the health management function may include changing display by the image display unit.

Here, “changing display” includes changing the size of characters, changing the color arrangement of a screen (including characters), and the like. With such a configuration, it is possible to present the information relating to health management in an appropriate display mode in accordance with the visual acuity of the user.

The information processing terminal may include an audio output unit, the health management function may include presenting information via the audio output unit, the indicator may include an indicator relating to hearing acuity of the user, and the setting unit setting a mode of providing the health management function may include changing output audio by the audio output unit.

Here, “changing output audio” includes changing the volume of the audio, changing the pitch of the audio, changing the playback speed of the audio, and the like. With such a configuration, it is possible to output an appropriate sound according to the hearing acuity of the user.

The health management function may include presenting to the user a method of performing exercise relating to treatment of a disease or injury of the user, the indicator may include an indicator relating to at least one of body temperature, blood pressure, pulse, and electrocardiogram of the user, and the setting unit setting a mode of providing the health management function may include changing content and/or intensity of the exercise or presenting information indicating that the exercise should be stopped.

With such a configuration, when it is determined that the user is in a poor physical condition (a fever state or the like) using biological information such as a body temperature as an indicator, information indicating that the exercise therapy should be stopped can be presented, or the exercise content or the intensity can be changed at an appropriate time and presented to the user.

The information processing system may further include an information processing apparatus configured to communicate with the information processing terminal, and the information processing apparatus may provide, to the information processing terminal, information of the indicator to be used in determining the suitability in accordance with content of the health management function to be provided by the application recording medium (or recording medium).

For example, in a case where there are a plurality of application recording mediums (or recording mediums) that provide a health management function and the functions provided by the respective application recording mediums are different from each other, the content of the indicator (that is, the user information to be acquired) necessary for the suitability determination of the user is different depending on the function to be provided. What user information is necessary to obtain a desired function is determined on the server (information processing apparatus) side, and the necessary user information is proposed to the user (information processing terminal), thereby providing a more user-friendly system.

Also, the information processing apparatus may issue authentication information corresponding to each user and each health management function and, in a case where the authentication information is received from the information processing terminal, may provide the information processing terminal with information of the indicator to be used in determining the suitability relating to the user and the health management function corresponding to the authentication information. In this manner, the server issues authentication information (access code) for each user and for each health management function, and the normal function is activated (enabled) on the condition that the user transmits the authentication information to the server by inputting the authentication information in the information processing terminal on the user side. In this manner, appropriate personal authentication and information management can be performed. In a case where it is desirable to use a health management function under the supervision of a physician, such as for treatment of a disease, it is desirable that the authentication information is transmitted to the user via the physician.

Another aspect of the present invention includes an information processing terminal configured to execute an application recording medium (or recording medium) for providing a health management function relating to management of health of a user, the information processing terminal including:
a user information acquisition unit configured to acquire user information including an indicator relating to health of the user;
a user suitability determination unit configured to perform a determination of suitability of the user for the health management function to be provided, on a basis of an indicator included in the user information; and
a setting unit configured to set a mode of providing the health management function according to a result of the determination.

Yet another aspect of the present invention includes a health management function providing method using an information processing terminal configured to execute an application recording medium (or recording medium) for providing a health management function relating to management of health of a user, the method including:
acquiring user information including an indicator relating to health of the user;
performing a determination of suitability of the user for the health management function to be provided, on a basis of an indicator included in the user information; and setting a mode of providing the health management function according to a result of the determination.

Another aspect of the present invention includes a recording medium (or recording medium) for causing a computer to execute each of the steps according to such a method and a computer-readable recording medium non-temporarily storing such a recording medium.

The configurations and processing described above can be combined with one another to constitute the present invention unless the combination leads to technical contradiction.

### Advantageous Effects of Invention

According to the present invention, a function can be provided in an appropriate manner in accordance with the characteristics or the state of a user when providing the function related to user health management to the user.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a schematic view illustrating a hardware configuration of an information processing terminal according to a first embodiment.

FIG. 2 is a block diagram illustrating a functional configuration of the information processing terminal according to the first embodiment.

FIG. 3 is a flowchart illustrating the flow of processing executed in the information processing terminal according to the first embodiment.

FIG. 4 is a schematic diagram illustrating an overview of an information processing system according to a second embodiment.

FIG. 5 is a block diagram illustrating a functional configuration of a user terminal according to the second embodiment.

FIG. 6 is a table illustrating the relationship between the use application and the indicators to be used in user suitability determination according to the second embodiment.

FIG. 7 is a block diagram illustrating a functional configuration of a server device according to the second embodiment.

FIG. 8 is a diagram illustrating information exchange and the flow of processing executed in the information processing system according to the second embodiment.

FIG. 9 is a schematic diagram illustrating an overview of an information processing system according to a third embodiment.

FIG. 10 is a block diagram illustrating a functional configuration of a server device according to the third embodiment.

FIG. 11 is a diagram illustrating information exchange and the flow of processing executed in the information processing system according to the third embodiment.

### DESCRIPTION OF EMBODIMENTS

### First Embodiment

Embodiments of the present invention will be specifically described below with reference to the drawings. However, it should be noted that the dimensions, shape, relative arrangement and the like of the components described in this embodiment are not intended to limit the scope of this invention to them alone, unless otherwise stated.

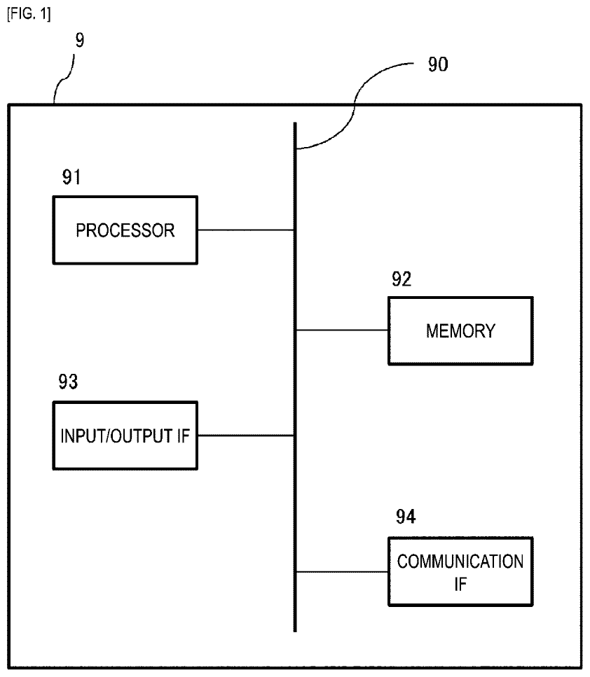

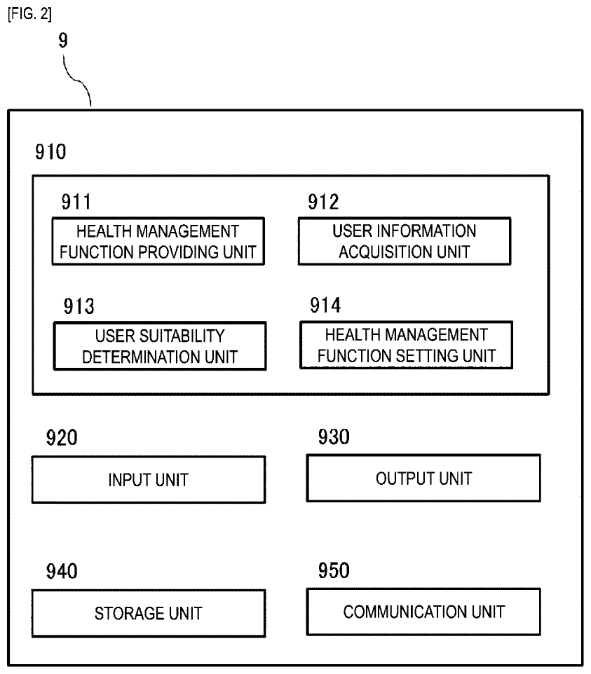

FIG. 1 is a schematic diagram schematically illustrating an apparatus configuration of an information processing terminal 9 according to the present embodiment. FIG. 2 is a block diagram schematically illustrating a functional configuration of the information processing terminal 9. The information processing terminal 9 is an information processing device configured to be capable of providing a function for health management of a user and may be, for example, a smartphone, but is not necessarily limited thereto, and may be a tablet terminal, a smart display, a wearable terminal, or the like.

The functions for health management provided by the information processing terminal 9 include functions that can be used by healthy people, such as daily exercise recording and nutrition management, but are not limited thereto, and also include functions related to medical practice for diseases and injuries of patients, such as blood pressure management of hypertensive patients and rehabilitation of patients with external injuries.

As illustrated in FIG. 1, the information processing terminal 9 includes a processor 91, a memory 92, an input/output interface (IF) 93, and a communication IF 94, which are connected to each other by a connection bus 90.

The processor 91, for example, may use a central processing unit (CPU), a micro-processing unit (MPU), a digital signal processor (DSP), or the like.

Although not illustrated, the memory 92 includes a main storage device such as a flash memory, a random access memory (RAM), or a read only memory (ROM), and an auxiliary storage device such as a solid state drive (SSD), an erasable recording mediummable ROM (EPROM), a flash memory, a USB memory, or a secure digital (SD) memory card. The memory 92 stores various types of information such as a recording medium (or recording medium) executed by the processor 91, data processed by the processor 91, operation setting information, and user information and user suitability determination criteria described below. The processor 91 executes a recording medium (or recording medium) stored in the memory 92, and accordingly, each configuration of the information processing terminal 9 is controlled, so that each functional unit that achieves a predetermined purpose can be realized.

The input/output IF 93 includes an input/output device such as a touch panel display and a speaker, for example. The communication IF 94 may have an appropriate configuration in accordance with the connection method (wired or wireless, connection standard, or the like) with a communication network for connection.

The information processing terminal 9 includes a control unit 910, an input unit 920, an output unit 930, a storage unit 940, and a communication unit 950 as main functional configurations.

The control unit 910 is implemented by the processor 91 and controls the entire information processing terminal 9. The control unit 910 can realize each functional unit of a health management function providing unit 911, a user information acquisition unit 912, a user suitability determination unit 913, and a health management function setting unit 914 by executing an application recording medium (or recording medium) related to health management. These will be described in detail below.

The input unit 920 transmits various input signals input via the input/output IF 93 to the control unit 910. The output unit 930 outputs various types of information to devices such as a touch panel display (not illustrated) and a speaker (not illustrated) via the input/output IF 93. The communication unit 950 performs information communication with other devices via the communication IF 94 (and a communication network). Examples of other devices include a biological information measurement apparatus described later.

The health management function providing unit 911 is implemented by executing a health management application recording medium (or recording medium) (hereinafter, the application recording medium is also simply referred to as an “application”) stored in the memory 92. For example, the health management function provided in the present embodiment is a function of “prompting rehabilitation exercise by displaying a video of a rehabilitation method in accordance with the site and movement of a rehabilitation patient.” The video may be displayed on the touch panel display of the information processing terminal 9 or may be output to another display device such as a television by communicating with the other display device.

The user information acquisition unit 912 acquires user information including an indicator related to the health of the user. The acquired user information may be stored in the storage unit 940 (memory 92). The user information here includes various information such as biological information such as blood pressure, pulse, body temperature, weight, and electrocardiogram; body function information such as eyesight, hearing, muscle strength, and cognitive function; attribute information such as age and gender; and symptoms (for example, pain, numbness, and the like) in the case of a disease or injury and the site, intensity, and the like thereof.

Specific modes of acquiring the user information include receiving a manual input from the user, receiving sensing information by a sensor, receiving a biological information measurement result by a biological information measurement device, reading information stored in the memory 92, and the like. When the information processing terminal 9 is a wearable terminal, the information processing terminal 9 may include a sensor (for example, an electrode for electrocardiogram measurement, a photoelectric sensor for pulse wave measurement, or the like) and a function of measuring biological information such as an electrocardiogram or blood pressure.

The user suitability determination unit 913 determines the suitability of the user for the provided health management function on the basis of the indicator included in the acquired user information. The “indicator” here is not limited to one and may be a plurality of indicators depending on the type of information. The indicator used for the determination of the suitability may be different depending on the health management function to be provided. Specifically, for the health management function provided in the present embodiment, the user suitability is determined using each indicator including cognitive function, body temperature, blood pressure, exercise capability (calculated from the number of steps and walking speed), hearing acuity, visual acuity, age, medical history, and pain (of the rehabilitation target site).

The user suitability determination performed by the user suitability determination unit 913 includes determination of whether or not the health management function can be used (hereinafter, also referred to as first determination) and determination of whether or not it is necessary to change the provision mode of the health management function (hereinafter, also referred to as second determination). However, only one of the determinations may be performed according to the content of the provided health management function.

The health management function setting unit 914 sets the mode of providing the health management function according to the determination result of the user suitability determination unit 913. The setting of the mode of providing the health management function includes setting a restriction on all or a part of the health management function. The “restriction” mentioned here is not limited to stopping the provision of the health management function until a certain condition is satisfied and includes adding annotation information (doubt about the accuracy of the information or the like) to the biological information in a case where the measured biological information is transmitted to the outside.

The setting of the mode of providing the health management function also includes optimizing the provision of the health management function in accordance with the user, such as changing the color arrangement of the display image, enlarging the display image (including text information), and changing the level of the standard output volume, in accordance with the body functions of the user.

In the present embodiment, the functional units of the user information acquisition unit 912, the user suitability determination unit 913, and the health management function setting unit 914 are realized by executing the user suitability determination application stored in the memory 92.

### Processing Flow

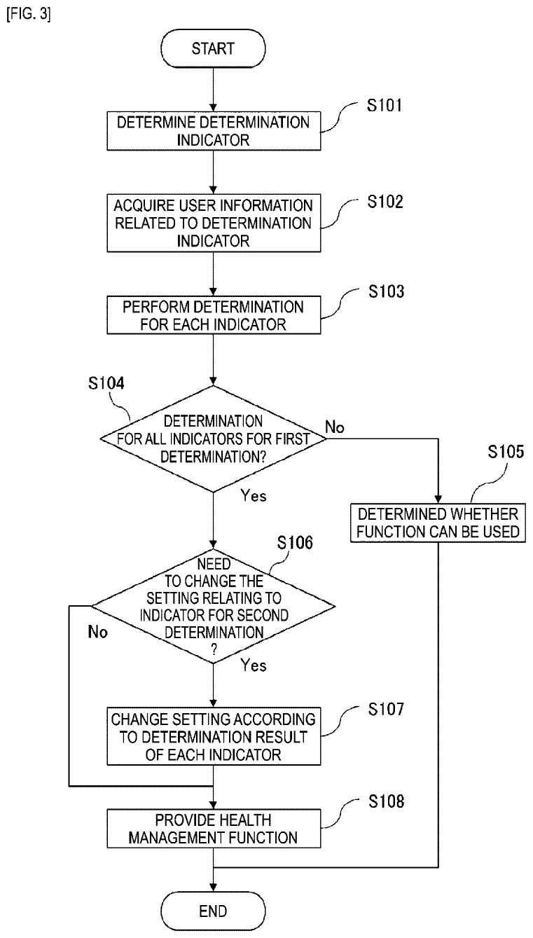

Next, the flow of processing executed in the information processing terminal 9 of the present embodiment will be described with reference to FIG. 3. FIG. 3 is a flowchart illustrating a flow of the processing related to providing the health management function performed by the information processing terminal 9.

As illustrated in FIG. 3, when the health management application and the user suitability determination application are activated, that is, read out from the auxiliary storage device and executed by the processor 91, the control unit 910 determines the indicator necessary for user suitability determination related to providing the health management function (S101). Then, the user information acquisition unit 912 acquires user information including the indicators determined in the S101 (S102).

As described above, in the present embodiment, the indicators used for the user suitability determination are cognitive function, body temperature, blood pressure, exercise capacity, hearing acuity, visual acuity, age, medical history, and pain. The method of acquiring each indicator may be different depending on the indicator, and for example, the user may be requested to manually input the age, medical history, and pain via a touch panel or the like. The body temperature and the exercise capability (determined by the number of steps and the walking ability) may be automatically acquired by a sensor included in the information processing terminal 9. The results of the measurement of the cognitive function, the blood pressure, the hearing acuity, and the visual acuity are acquired. The measurement results may be acquired from another device such as a blood pressure monitor via a communication unit or by allowing the user to manually input the measurement results, or the measurement results may be acquired for an indicator for which the information processing terminal 9 has a function for measurement. The cognitive function may be measured by another subroutine (such as a medical interview phase), and, for example, a known cognitive function examination method as disclosed in JP 2020-151045 A may be used.

Next, the user suitability determination unit 913 determines whether or not a condition corresponding to a change in the provision mode of the health management function is satisfied for each of the indicators included in the user information acquired in S102 (S103). Then, in S104, the user suitability determination unit 913 determines whether or not all the indicators for the first determination satisfy the condition for “can” provide health management function F1 (S104). In the present embodiment, it is determined whether or not the indicators of the cognitive function (whether a level that can use the health management function or not), body temperature (fever or not, for example, less than 37.5° C.), blood pressure (equal to or less than a predetermined value or not) all satisfy the conditions.

In S104, in a case where the user suitability determination unit 913 determines that any one of the indicators does not satisfy the condition, the processing proceeds to S105, and it is determined to prohibit the provision of the health management function (S105), and the series of processing ends. This prevents the health management function from being provided until some stopping condition is removed. When such processing is executed, information such as the reason why the health management function cannot be provided and the condition for resuming the provision of the function may be presented to the user by displaying the information on the touch panel display.

On the other hand, in S104, in a case where all the indicators related to the first determination satisfy the condition, the processing proceeds to S106. In S106, the user suitability determination unit 913 determines whether or not it is necessary to change the provision mode of the health management function with respect to each of the indicators for the second determination (S106). Here, in a case where it is determined that the mode of providing the health management function does not need to be changed, the processing proceeds to S108, the health management function is provided by the health management function providing unit 911 (in the present embodiment, the rehabilitation method is displayed as a video), and the series of processing ends.

On the other hand, in S106, in a case where it is determined that a change is required to the mode of providing the health management function, the processing proceeds to S107. In S107, the health management function setting unit 914 changes the provision mode of the health management function from the default content in accordance with the indicator and the content thereof determined to be required to be changed for the provision mode of the health management function in S106 (S107).

Note that in the second determination, for example, the following determination is performed. That is, it is determined whether or not it is necessary to change the intensity of the exercise illustrated in the video on the basis of the indicator of the motor function. It is determined whether it is necessary to change the volume of the video on the basis of the hearing acuity indicator. It is determined whether or not the size or color arrangement of the characters needs to be changed on the basis of the visual acuity indicator. It is determined whether it is necessary to change the playback speed of the video on the basis of the age indicator. It is determined whether or not the video playback length or the exercise intensity needs to be changed on the basis of the medical history indicator (for example, whether or not there is a medical history indicating that it is better to avoid intense exercise such as abdominal aortic aneurysm). It is determined whether or not it is necessary to change the video to be displayed (change to a video with content that avoids exercise involving moving a site with pain) on the basis of the pain indicator (for example, whether or not there is pain in a place different from a site to be rehabilitated).

In S107, the health management function setting unit 914 changes the mode of providing the health management function (such as the content of the video) in accordance with the determination result as described above. The health management function providing unit 911 provides a health management function in which such a change is reflected (S108), and the series of processing ends.

According to the information processing terminal 9 of the present embodiment as described above, it is possible to restrict the provision of the function to a user in a state where it is not appropriate to provide the health management function. In addition, it is possible to provide the function by optimizing the mode of providing the health management function in accordance with the state of the user. Thus, it is possible to reduce the possibility of a wrong treatment action being caused by providing the health management function in a situation without the supervision of a medical professional, and it is possible to provide a tool of health management with higher convenience for the user.

### Second Embodiment

### System Configuration

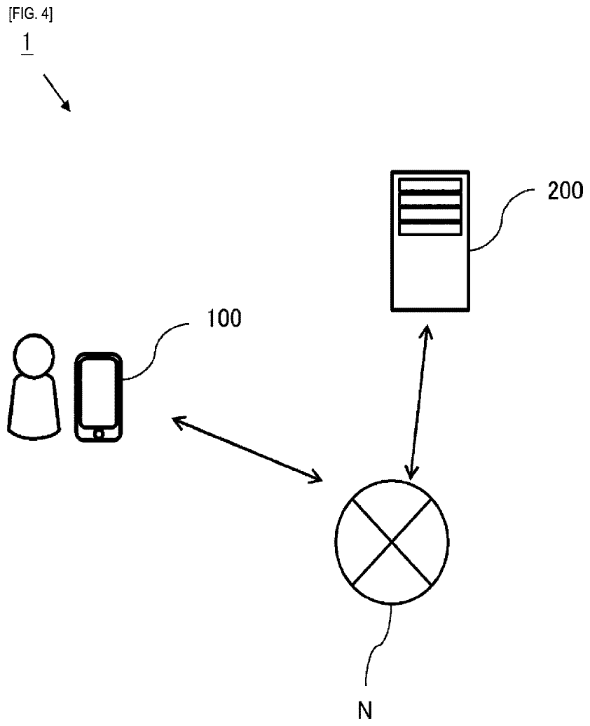

Next, another embodiment of the present invention will be described. FIG. 4 is a schematic diagram illustrating a configuration of an information processing system 1 according to the present embodiment. As illustrated in FIG. 4, the information processing system 1 according to the present embodiment includes an information processing terminal (hereinafter referred to as a user terminal) 100 used by a user and a server device 200, and these components can communicate with each other via a communication network N. Although not illustrated, the information processing system 1 may include a medical institution terminal used by a medical professional such as a physician and a biological information measurement device (a blood pressure monitor, a body composition meter, or the like) having a communication function.

### User Terminal

The user terminal 100 may be a smartphone as with the information processing terminal 9 of the first embodiment, but is not necessarily limited thereto, and may be a tablet terminal, a smart display, a wearable terminal, or the like. That is, the hardware configuration of the user terminal 100 according to the present embodiment can be the same as that of the information processing terminal 9 of the first embodiment, and thus the hardware configuration will not be described again.

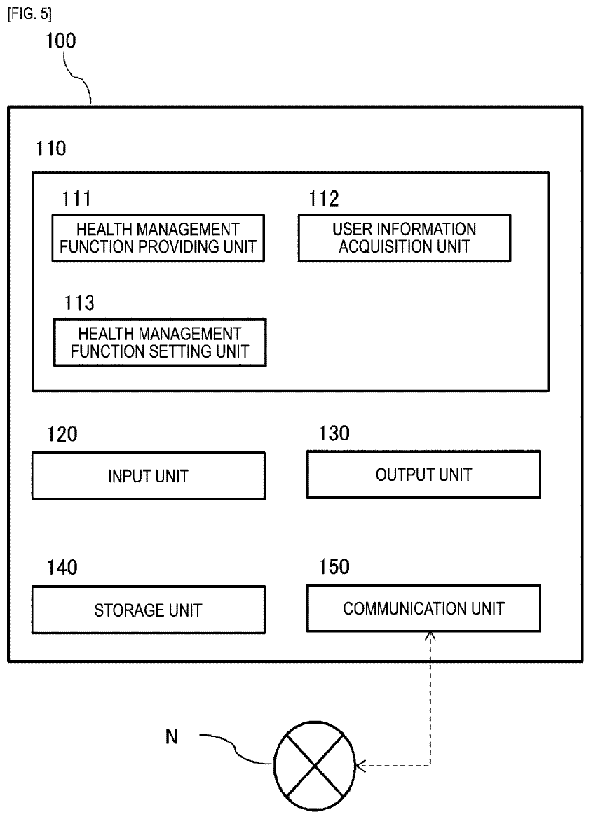

FIG. 5 is a block diagram illustrating functional blocks of the user terminal 100. As illustrated in FIG. 5, the user terminal 100 includes a control unit 110, an input unit 120, an output unit 130, a storage unit 140, and a communication unit 150 as main functional components. The user terminal 100 in the present embodiment has a functional configuration substantially similar to that of the information processing terminal 9 in the first embodiment. That is, the control unit 110, the input unit 120, the output unit 130, the storage unit 140, and the communication unit 150 of the user terminal 100 correspond to the control unit 910, the input unit 920, the output unit 930, the storage unit 940, and the communication unit 950 of the information processing terminal 9, respectively.

The functional units of a health management function providing unit 111, a user information acquisition unit 112, and a health management function setting unit 113 included in the control unit 110 are the same as the corresponding functional units of the information processing terminal 9. However, the present embodiment is different from the information processing terminal 9 of the first embodiment in that the control unit 110 does not include a user suitability determination unit.

In the present embodiment, the health management function providing unit 111 can provide a plurality of different health management functions. That is, a plurality of different health management applications are installed in the user terminal 100, and different health management functions are provided according to the executed application. Hereinafter, a case where three applications, that is, a health management application A, a health management application B, and a health management application C, are installed will be described.

Note that the health management application A provides a function of “prompting rehabilitation exercise by displaying a rehabilitation method in accordance with a site and movement of a patient to be rehabilitated as a video” (hereinafter also referred to as a “first health management function”), as described in the first embodiment. On the other hand, the health management application B provides a function of “performing cognitive function evaluation using vision and language for determining an activity resumption time after a concussion from sports or the like” (hereinafter also referred to as a “second health management function”). The health management application C provides a function of “performing blood pressure lowering treatment by improving lifestyle habits or drug therapy” (hereinafter also referred to as a “third health management function”).

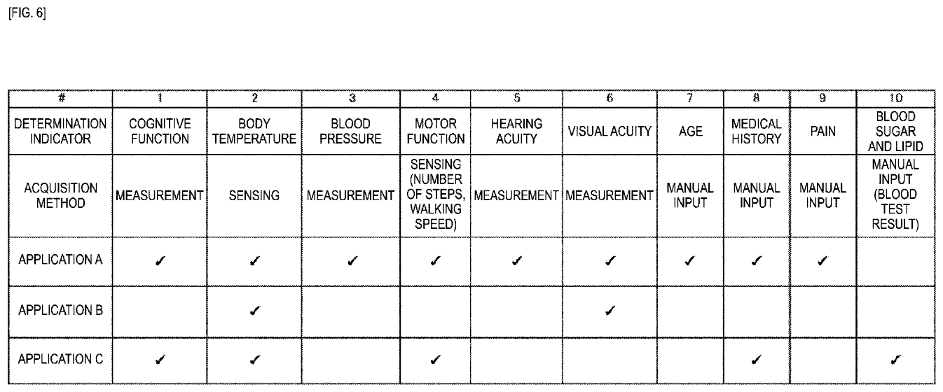

The first to third health management functions provided by the health management applications A to C are provided with different user suitability determination indicators (and criteria) according to the difference in function. FIG. 6 illustrates a list of the relationships between the indicators used for user suitability determination and the acquisition methods thereof for the functions provided by the respective applications.

Referring to the table of FIG. 6, it can be seen that the indicators for user suitability determination for the first health management function provided by the health management application A are “cognitive function, body temperature, blood pressure, motor function, hearing acuity, visual acuity, age, medical history, and pain”. On the other hand, it can be seen that the indicators for the second health management function provided by the health management application B are “body temperature and visual acuity”, and the indicators for the third health management function provided by the health management application C are “cognitive function, body temperature, motor function, medical history, and blood sugar and lipid”.

Further, according to the table of FIG. 6, the acquisition methods of the respective indicators are also different from each other. Specifically, the respective indicators of “cognitive function, blood pressure, hearing acuity, and visual acuity” are acquired by measurement. The “body temperature and motor function” are acquired by sensing. The “age, medical history, pain, blood sugar and lipid” are acquired by manual input by the user. Note that, regarding the measurement, the measurement result may be acquired in cooperation with another biological information measurement apparatus, or the user terminal 100 itself may have a configuration for the measurement. In addition, regarding sensing, the user terminal 100 may include a temperature sensor and an acceleration sensor and acquire information. Description of the measurement, sensing, and manual input that are redundant with respect to the description in the first embodiment will be omitted.

### Server Device

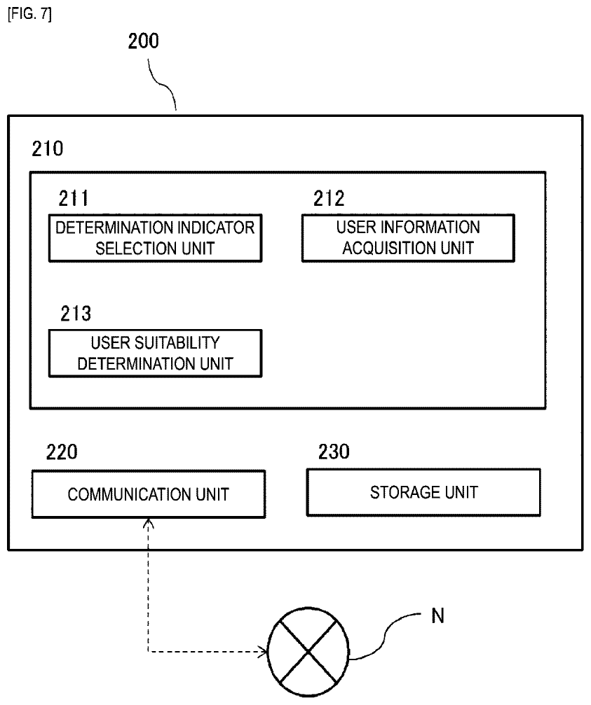

The server device 200 has the role of supporting processing when the health management function is provided in the user terminal 100 in communication cooperation with the user terminal 100. FIG. 7 is a block diagram illustrating a functional configuration of the server device 200. A typical server computer can be used as the server device 200. That is, although not illustrated, the server device 200 is an information processing apparatus including a processor, a memory, an input/output IF, a communication IF, and the like as a hardware configuration. As illustrated in FIG. 7, the server device 200 includes functional blocks of a control unit 210, a communication unit 220, and a storage unit 230. Although not illustrated, an operation input unit and an output unit may be provided.

The control unit 210 controls the server device 200 and is implemented by a processor such as a CPU, for example. The control unit 210 includes, as functional modules, functional units of a determination indicator selection unit 211, a user information acquisition unit 212, and a user suitability determination unit 213.

The communication unit 220 includes a hardware configuration configured to connect the server device 200 to the communication network N (a communication interface board, a wireless communication circuit for wireless communication, or the like).

Although not illustrated, the storage unit 230 includes a main storage device such as a ROM or a RAM and an auxiliary storage device such as an EPROM, an HDD or an SSD, or removable media. The auxiliary storage device stores an operating system, various recording mediums, and the like.

The determination indicator selection unit 211 selects an indicator to use in user suitability determination according to the type of the health management function provided by the user terminal 100. As illustrated in FIG. 6, the indicator to be used for determining the user suitability differs depending on the health management function to be provided. Thus, in the user terminal 100 in which a plurality of health management applications can be used, different user information is required for each health management function (that is, for each application to be used). The determination indicator selection unit 211 acquires information on which health management application is used in the user terminal 100 (that is, which health management function the user desires to be provided) and determines an indicator for user suitability determination according to the health management application to be used on the basis of the information. Specifically, for example, as illustrated in the table of FIG. 6, a data table in which the health management application (the health management function provided by the health management application) and the indicator necessary for the user suitability determination are associated with each other is held in the storage unit 230, and the health management application can be determined by referring to the data table.

The user information acquisition unit 212 acquires user information of the user using the user terminal 100 via the communication unit 220. More specifically, the user information including the indicator determined by the determination indicator selection unit 211 is acquired.

The user suitability determination unit 213 determines the suitability of the user for the health management function provided in the user terminal 100 on the basis of the indicator included in the acquired user information. As described above, since the health management function provided in the user terminal 100 differs depending on the health management application to be used, the user suitability determination unit 213 performs determination according to each application. In addition, the user suitability determination performed by the user suitability determination unit 213 includes the determination (first determination) of whether or not the health management function can be used and the determination (second determination) of whether or not it is necessary to change the provision mode of the health management function, as in the case of the first embodiment, but only one of the determinations may be performed depending on the content of the provided health management function. As described above, in the present embodiment, the function of the user suitability determination unit 213 is realized in the server device 200, not in the user terminal 100.

### Processing Flow

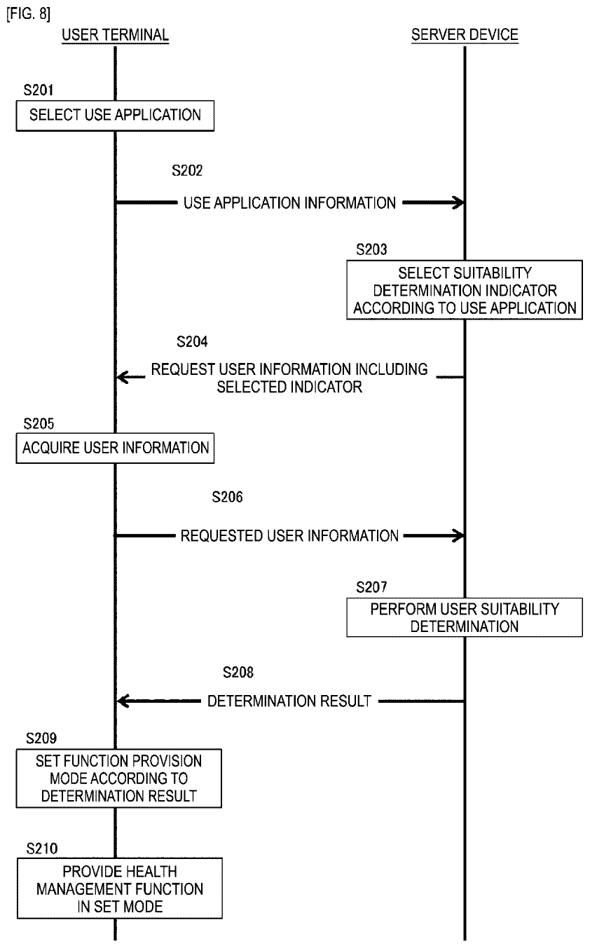

Next, a flow of the processing executed by the information processing system 1 according to the present embodiment having the above-described configuration will be described with reference to FIG. 8. FIG. 8 is a diagram illustrating the flow of information exchange and processing executed in the information processing system 1. As illustrated in FIG. 8, first, when the user executes the health management application with the function the user desires to use in the user terminal 100 (S201), information indicating which health management application is executed is transmitted from the user terminal 100 to the server device 200 via the communication network N (S202). At this time, attribute information of the user (age, gender, user ID, and the like) may be transmitted together.

In the server device 200 that has received the information on the health management application to be used, the determination indicator selection unit 211 selects a suitability determination indicator according to the application (S203). Next, the server device 200 transmits a transmission request for information on the one or more determination indicators selected in S203 and user information including the determination indicators to the user terminal 100 via the communication network N (S204).

Next, the user terminal 100 executes processing for acquiring user information including the indicator requested in S204 (S205). Note that for an indicator that requires manual input or measurement, necessary user information is acquired by, for example, displaying a user interface (UI) for information acquisition on a display to receive an input from the user or prompt measurement. Then, the user terminal 100 transmits the user information acquired in S205 to the server device 200 (S207).

In S207, in the server device 200 that has received the user information including the indicator necessary for the user suitability determination, the user suitability determination unit 213 determines the user suitability according to the health management application used in the user terminal (S207). For example, when the health management application B is used in the user terminal 100, the user suitability determination unit 213 performs only the first determination using the body temperature and the visual acuity indicators. Specifically, when the health management application B is executed by the user terminal 100, the user suitability determination unit 213 determines whether or not there is fever and whether or not there is eye movement capability or a field of view in which the second health management function can be used, and determines only whether or not the function can be used.

On the other hand, when the health management application C is used in the user terminal 100, the user suitability determination unit 213 performs the first determination based on the indicators of “cognitive function (if cognitive function is reduced, there is a risk that they will forget the fact that they took medicine and overdose) and body temperature” and further performs the second determination for the indicators of “motor function, medical history, and blood sugar and lipid”.

More specifically, whether or not to change the exercise intensity (how to change the exercise intensity if the exercise intensity is changed) is determined from the walking speed across several days using the motor function indicator. In addition, whether or not the exercise intensity needs to be changed (the degree of change in the case of change) and whether or not to propose lifestyle improvement other than exercise are determined using the medical history indicator (for example, whether or not there is a medical history indicating it is better to avoid intense exercise such as abdominal aortic aneurysm). In addition, it is determined whether or not to propose a diet improvement (for example, presentation of food containing a large amount of cholesterol and proposal of intake restriction) using the blood sugar and lipid indicator (blood glucose level, LDL cholesterol level, and the like).

When the determination by the user suitability determination unit 213 is completed, the server device 200 transmits the determination result to the user terminal (S208). Next, in the user terminal 100, the health management function setting unit 113 performs setting so as to reflect the provision mode of the function according to the transmitted determination result (S209). The “provision mode of the function” mentioned here includes stopping the provision of the function itself and limiting the function to be provided. Thereafter, the health management function providing unit 111 provides the health management function by the application used by the user in the mode set in S209 (S210).

According to the information processing system 1 of the present embodiment as described above, even when there are many types of health management functions that can be provided by the user terminal 100, it is possible to reduce the load (calculation load, storage capacity, and the like) on the user terminal 100 side by determining the user suitability in the server device 200 having high processing capacity.

### Third Embodiment

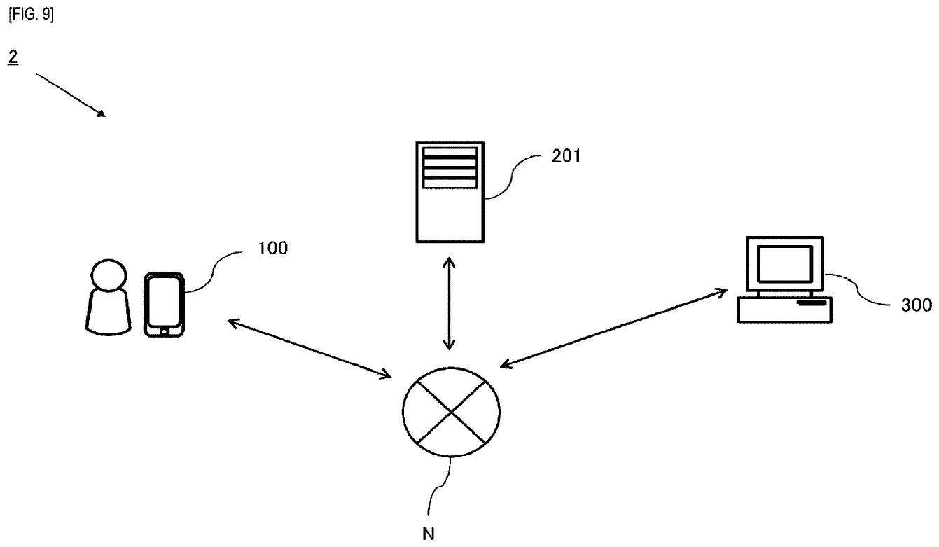

The information processing system may be configured to be communicably connected to a medical institution terminal used by a medical professional. FIG. 9 illustrates a schematic configuration of an information processing system 2 according to such an embodiment. In the present embodiment, configurations, processing, and functions that are similar to those in the embodiments described above use the same names and reference numerals and are not described again. As illustrated in FIG. 9, the information processing system 2 according to the present embodiment includes the user terminal 100, a server device 201, and a medical institution terminal 300, and these are communicably connected via the communication network N. Note that the user terminal 100 is configured similarly to that described in the second embodiment and will thus not be described in detail.

The medical institution terminal 300 may be a general-purpose computer including a processor, a memory, an input/output IF, a communication IF (none of which are illustrated), and the like. The medical institution terminal 300 may cooperate with an electronic medical chart or may have a function of supporting medical practice by a physician by displaying data of a patient in an easy to understand manner.

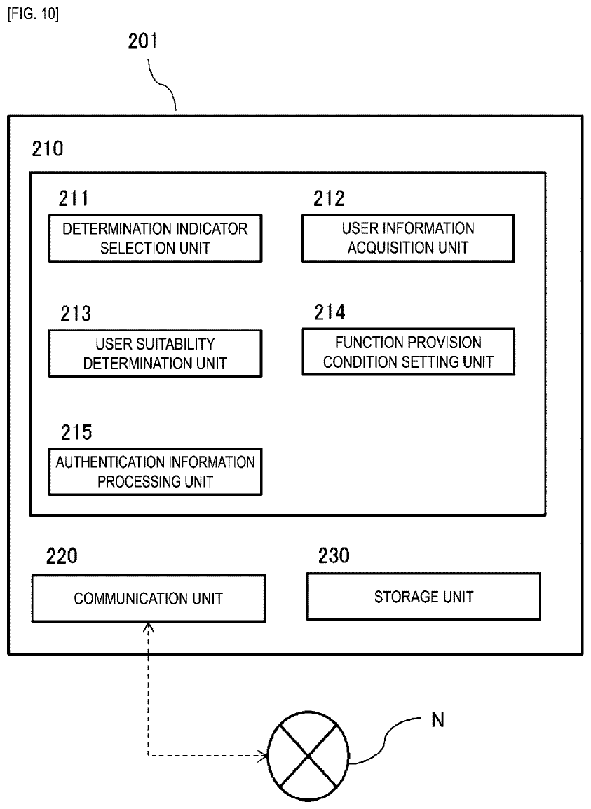

As described in the second embodiment, the server device 201 is an information processing apparatus including a processor, a memory, an input/output IF, a communication IF, and the like as a hardware configuration. FIG. 10 is a block diagram illustrating the functional configuration of the server device 201 according to the present embodiment. As illustrated in FIG. 10, the server device 201 according to the present embodiment is the same as the server device 200 according to the second embodiment except that the server device 201 further includes a function provision condition setting unit 214 and an authentication information processing unit 215 as functional units.

The function provision condition setting unit 214 sets a function provision condition for each user and for each health management function (application) on the basis of information provided from the medical institution terminal 300. Here, the information provided from the medical institution terminal 300 is information input by a medical professional such as a physician and includes a type of a health management function (application) used by a patient (that is, a user), medical practice information of the patient, and some user information such as attribute information of the patient. The function provision condition includes a period during which the health management function provided by the user terminal 100 is valid (or expiration date), an indicator selected by the determination indicator selection unit, and the like.

The authentication information processing unit 215 issues an access code for enabling the health management function provided in the user terminal 100 on the basis of the information provided from the medical institution terminal 300 and the function provision condition set by the function provision condition setting unit 214. The access code is unique for each combination of the user and the type of the health management function (and the expiration date which the health management function can be used until as necessary), and may be, for example, a character string, an encoded two dimensional code, or the like. The issued access code is transmitted to the medical institution terminal 300 via the communication unit 220, for example.

In addition, in a case where an access code is input in the user terminal 100, the authentication information processing unit 215 receives the access code via the communication network N, performs authentication, and executes processing of enabling the health management function in the user terminal 100.

### Processing Flow

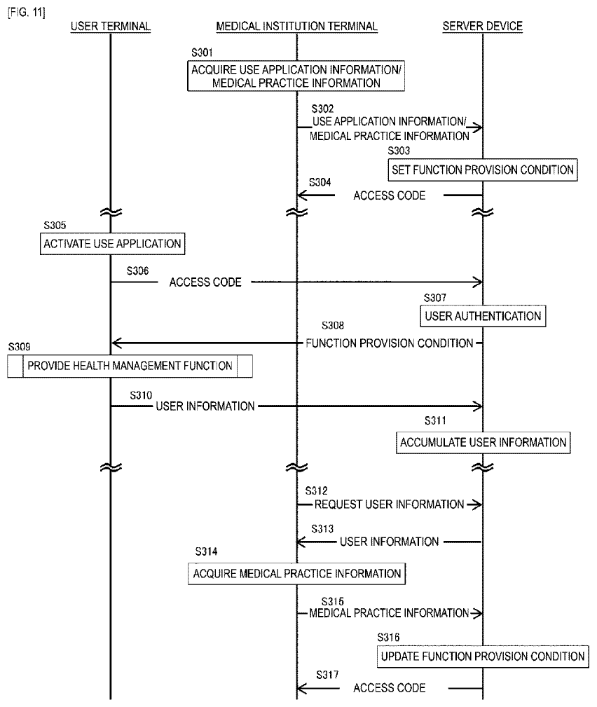

Among the health management functions available in the user terminal 100, for example, the “function of performing blood pressure lowering treatment by improving lifestyle habits or drug therapy” (third health management function) provided by the health management application C basically requires diagnosis or prescription by a physician. In a case where such a function is provided by the user terminal 100, it is desirable to enable the function in the user terminal 100 under the supervision of a physician on the premise of examination and prescription by the physician. Hereinafter, a flow of processing executed in the information processing system 2 according to the present embodiment will be described using an example where the third health management function is provided. FIG. 11 is a diagram illustrating the flow of information exchange and processing executed in the information processing system 2.

First, when a patient receives a medical examination in a medical institution, a physician determines a treatment strategy of performing health management by the health management application C on the patient and inputs information such as the type of health management function (application) to be used, an expiration date of the health management function, medical practice information of the patient, and some user information such as attribute information of the patient to the medical institution terminal 300. That is, the medical institution terminal 300 acquires the information on the applications to be used and the patient information such as the medical practice information (S301). The expiration date of the health management function may be, for example, a period of time obtained by adding a certain margin to the next scheduled medical examination date. The medical practice information may include, for example, the state of the patient's symptoms, the content of the medical interview, the prescription, the scheduled date of the next medical examination, and the like. In addition, the indicators and/or thresholds for user suitability determination included in the function provision condition described later may be designated by a physician in the S301.

Next, the medical institution terminal 300 transmits the information acquired in S301 to the server device 201 via the communication unit (not illustrated) and the communication network N (S302). The server device 201 sets a condition (function provision condition) for providing the third health management function in the user terminal 100 of the target patient using the received information such as the information of the application to use and the medical practice information and issues an access code reflecting the set condition (S303). Specifically, the function provision condition setting unit 214 sets a condition including the indicator (and the determination threshold) selected by the determination indicator selection unit 211, the determination indicator (and the determination threshold) input by a physician, or the like, and the authentication information processing unit 215 issues an access code. The condition mentioned here may include an expiration date which the health management function can be used until.

The server device 201 transmits the access code to the medical institution terminal 300 via the communication network N (S304). The access code received by the medical institution terminal 300 is output by a physician by a method such as printing and is transmitted to the patient using the user terminal 100.

After the patient finishes the medical examination at the medical institution, the patient performs a blood pressure lowering treatment by using the third health management function provided in the user terminal 100 used by the patient according to an instruction of the physician. To be specific, the health management application C is started up in the user terminal 100 (S305). Here, the health management application C is configured to require an access code to be input when the health management application C is used for the first time, and the third health management function is not provided unless an appropriate access code is input.

The patient inputs the access code using an input unit (for example, a touch panel display or a camera) of the user terminal 100. The input access code is transmitted to the server device 201 via the communication network N (S306), and the authentication information processing unit 215 executes authentication processing of the access code (S307). When the authentication is properly performed, the function provision condition associated with the access code input by the patient is transmitted from the server device 201 to the user terminal (S308).

When the user terminal 100 receives the function provision condition, the health management function providing unit 111 provides the function on the basis of the condition (S309). Note that, although processing such as user suitability determination is also executed when providing the health management function, each item of processing when providing such functions is similar to the processing of S204 onward in the information processing system 1 of the second embodiment, and thus the description thereof will be omitted here. Since the third health management function includes a content of the function collecting and accumulating the daily vitals data of the user, the information acquired by the user information acquisition unit 112 of the user terminal 100 is transmitted to the server device 201 via the communication network N (S310) and is accumulated in the storage unit 230 (S311).

Then, at the time of the next medical examination (or in preparation for the next medical examination), the physician requests the transmission of the user information stored in the server device 201 from the medical institution terminal 300 (S312), in order to refer to the user information including vitals data of the patient. The server device 201 that has received the request transmits the requested user information to the medical institution terminal 300 (S313). Before the user information is transmitted, authentication processing such as requesting input of an access code or certain user information may be executed.

The physician examines the patient by using the information provided from the server device 201 and inputs the medical practice information, the next expiration date of the health management application, and the like to the medical institution terminal 300. That is, the medical institution terminal 300 acquires the medical practice information (S314). The medical institution terminal 300 transmits the acquired medical practice information to the server device 201 via the communication network N (S315). In the server device 201, the function provision condition setting unit 214 updates the function provision condition of the health management function provided to the target patient on the basis of the received medical practice information (S316). The update mentioned here includes continuously setting the same condition as the previous time. Then, the authentication information processing unit 215 newly issues an access code associated with the updated function provision condition and transmits the access codes to the medical institution terminal 300 (S317). The subsequent processing is similar to that of S305 onward.

According to the information processing system 2 as described above, it is possible to provide a health management function that enables a patient to live a life (including taking a medicine) in a manner appropriate for treatment without requiring a medical professional to perform daily care for a disease that can be treated by improving lifestyle habits or by drug therapy under the supervision of a physician on the premise of medical practice by a physician. In addition, a physician can check the transition of the daily vitals data of the patient at any timing such as before the patient's examination, and the quality of the medical practice can be improved.

### Modified Example

In the case where the health management function as in the second embodiment is provided, when the cognitive function of the patient has decreased, there is a doubt about the accuracy of the daily vitals data transmitted from the user terminal 100 to the server device 201, which may adversely affect the diagnosis of the physician. Thus, in the case of providing the third health management function, when it is determined that the cognitive function of the user has decreased but not to the extent that the provision of the function should be stopped immediately in the determination by the user suitability determination unit 213, the health management function setting unit 113 of the user terminal 100 may be set to output a warning message encouraging early diagnosis by a physician.

When the patient receives a diagnosis from a physician within a predetermined period, the medical institution terminal 300 may transmit medical practice information to the server device 201 to update the function provision condition and issue an access code that can reset the provision mode of the health management function outputting the warning message. On the other hand, when the physician does not make a diagnosis within a predetermined period, the health management function setting unit 113 may set the provision mode so as to stop the provision of the function itself.

In this manner, even if there is a hospital visit with a physician scheduled in advance, it is possible to avoid a situation in which the patient is diagnosed by the physician regardless of the schedule, and thus, inaccurate vitals data of the patient is continuously acquired for a long period of time or acquisition of vitals data is omitted.

Alternatively, when it is determined that the cognitive function has decreased, additional information indicating that the cognitive function has decreased may be transmitted together with the user information transmitted to server device 201. In this case, the physician can make a diagnosis based on the possibility that the transmitted vitals data is not accurate, and thus it is possible to reduce the possibility that an erroneous medical practice is performed.

### Supplemental Note

The description of each example described above is merely illustrative of the present invention, and the present invention is not limited to the specific embodiments described above. Various modifications and combinations may be made within the scope of the technical idea of the present invention. For example, in the information processing terminal 9 according to the first embodiment, a plurality of health management applications may be available as in the second embodiment. That is, as in the second embodiment, the user information (indicator) to be acquired and the content of the user suitability determination may be different depending on the health management application to be executed.

In the information processing system 2 according to the third embodiment, the transmission destination of the user information (particularly, vitals data) transmitted from the user terminal 100 as a function of the health management application may be the medical institution terminal 300 instead of the server device 201. Alternatively, the user information may be transmitted to both the server device 201 and the medical institution terminal 300.

In the above-described embodiments, the health management application is stored in the storage unit of the terminal used by the user. However, the health management function may be provided in the form of SaaS (Software as a Service) via a network. In the first embodiment, the health management application and the user suitability determination application are described as separate application recording mediums, but the functional units of the information processing terminal 9 may be realized by an integrated application recording medium including the health management application and the user suitability determination application. In addition, in the second embodiment (and the modified example thereof), a configuration in which one user terminal and one server device are provided has been described, but a plurality of user terminals (and medical institution terminals) may be communicably connected to one server device.

Although the inventive concepts of the present disclosure have been described and illustrated with respect to example embodiments thereof, it is not limited to the example embodiments disclosed herein and modifications may be made therein without departing from the scope of the inventive concepts.

### REFERENCE SIGNS LIST

1, 2 Information processing system
9 Information processing terminal
90 Bus
91 Processor
92 Memory
93 Input/output IF
94 Communication IF
100 User terminal
200, 201 Server device
300 Medical institution terminal
110, 210, 910 Control unit
120, 230, 940 Storage unit
150, 220, 950 Communication unit
120, 920 Input unit
130, 930 Output unit
N Communication network

What is claimed is:

1. An information processing system at least including an information processing terminal configured to execute an application recording medium for providing a health management function relating to management of health of a user, the information processing system comprising:
a user information acquisition unit configured to acquire user information including an indicator relating to health of the user including at least a cognitive function of the user;
a user suitability determination unit configured to perform a determination of suitability of the user for the health management function to be provided, on a basis of an indicator included in the user information; and
a setting unit configured to set a mode of providing the health management function according to a result of the determination, wherein
setting a mode of providing the health management function performed by the setting unit includes presenting, when providing the health management function, information encouraging early examination by a physician in accordance with the suitability determined on a basis of an indicator relating to the cognitive function.

2. The information processing system according to claim 1, wherein
in a case where a mode of providing the health management function is set by the setting unit to present information encouraging early examination by a physician, and information indicating that an examination by a physician has been made is not input within a predetermined time period, the setting unit further sets a mode of providing a health management function to stop provision of the health management function.

3. The information processing system according to claim 1, wherein
the information processing terminal includes a communication unit,
the health management function includes transmitting the user information via the communication unit, and
the setting unit setting a mode of providing the health management function includes setting to add the result of the determination for the suitability to the user information and transmit the user information.

4. An information processing system at least including an information processing terminal configured to execute an application recording medium for providing a health management function relating to management of health of a user, the information processing system comprising:
a user information acquisition unit configured to acquire user information including an indicator relating to health of the user including at least a cognitive function of the user;
a user suitability determination unit configured to perform a determination of suitability of the user for the health management function to be provided, on a basis of an indicator included in the user information; and
a setting unit configured to set a mode of providing the health management function according to a result of the determination, wherein
the information processing terminal includes an image display unit,
the health management function includes presenting information via the image display unit,
the indicator includes an indicator relating to visual acuity of the user, and
the setting unit setting a mode of providing the health management function includes changing at least one of a size of characters displayed and color arrangement displayed by the image display unit in accordance with the suitability determined on a basis of an indicator relating to the visual acuity.

5. An information processing system at least including an information processing terminal configured to execute an application recording medium for providing a health management function relating to management of health of a user, the information processing system comprising:
a user information acquisition unit configured to acquire user information including an indicator relating to health of the user including at least a cognitive function of the user;
a user suitability determination unit configured to perform a determination of suitability of the user for the health management function to be provided, on a basis of an indicator included in the user information; and
a setting unit configured to set a mode of providing the health management function according to a result of the determination, wherein
the information processing terminal includes an audio output unit,
the health management function includes presenting information via the audio output unit,
the indicator includes an indicator relating to hearing acuity of the user, and
the setting unit setting a mode of providing the health management function includes changing at least one of volume of audio, pitch of audio, and playback speed of audio output by the audio output unit in accordance with the suitability determined on a basis of an indicator relating to the hearing acuity.

6. An information processing system at least including an information processing terminal configured to execute an application recording medium for providing a health management function relating to management of health of a user, the information processing system comprising:
a user information acquisition unit configured to acquire user information including an indicator relating to health of the user including at least a cognitive function of the user;
a user suitability determination unit configured to perform a determination of suitability of the user for the health management function to be provided, on a basis of an indicator included in the user information; and
a setting unit configured to set a mode of providing the health management function according to a result of the determination, wherein
the health management function includes presenting to the user a method of performing exercise relating to treatment of a disease or injury of the user,
the indicator includes an indicator relating to at least one of body temperature, blood pressure, pulse, and electrocardiogram of the user, and
the setting unit setting a mode of providing the health management function includes presenting information indicating that the exercise should be stopped in accordance with the suitability.

7. The information processing system according to claim 1, further comprising:
an information processing apparatus configured to communicate with the information processing terminal, wherein
the information processing apparatus provides, to the information processing terminal, information of the indicator to be used in determining the suitability in accordance with content of the health management function to be provided by the application recording medium.

8. The information processing system according to claim 3, further comprising:
an information processing apparatus configured to communicate with the information processing terminal, wherein
the information processing apparatus provides, to the information processing terminal, information of the indicator to be used in determining the suitability in accordance with content of the health management function to be provided by the application recording medium.

9. The information processing system according to claim 7, wherein
the information processing apparatus issues authentication information corresponding to each user and each health management function and, in a case where the authentication information is received from the information processing terminal, provides the information processing terminal with information of the indicator to be used in determining the suitability relating to the user and the health management function corresponding to the authentication information.

10. An information processing terminal configured to execute an application recording medium for providing a health management function relating to management of health of a user, the information processing terminal comprising:
a user information acquisition unit configured to acquire user information including an indicator relating to health of the user including at least a cognitive function of the user;
a user suitability determination unit configured to perform a determination of suitability of the user for the health management function to be provided, on a basis of an indicator included in the user information; and
a setting unit configured to set a mode of providing the health management function according to a result of the determination, wherein
the setting unit setting a mode of providing the health management function includes presenting information encouraging early examination by a physician when providing the health management function in accordance with the suitability determined on a basis of an indicator relating to the cognitive function.

11. A health management function providing method using an information processing terminal configured to execute an application recording medium for providing a health management function relating to management of health of a user, the method comprising:
acquiring user information including an indicator relating to health of the user including at least a cognitive function of the user;
performing a determination of suitability of the user for the health management function to be provided, on a basis of an indicator included in the user information; and
setting a mode of providing the health management function according to a result of the determination, wherein
the setting of a mode of providing the health management function includes presenting information encouraging early examination by a physician when providing the health management function in accordance with the suitability determined on a basis of an indicator relating to the cognitive function.

12. A health management function providing method using an information processing terminal configured to execute an application recording medium for providing a health management function relating to management of health of a user, the method comprising:
acquiring user information including an indicator relating to health of the user;
performing a determination of suitability of the user for the health management function to be provided, on a basis of an indicator included in the user information; and
setting a mode of providing the health management function according to a result of the determination, wherein
the information processing terminal includes an image display unit,
the health management function includes presenting information via the image display unit,
the indicator includes an indicator relating to visual acuity of the user, and
the setting of a mode of providing the health management function includes changing at least one of a size of characters displayed and color arrangement displayed by the image display unit in accordance with the suitability determined on a basis of an indicator relating to the visual acuity.

13. A health management function providing method using an information processing terminal configured to execute an application recording medium for providing a health management function relating to management of health of a user, the method comprising:
acquiring user information including an indicator relating to health of the user;
performing a determination of suitability of the user for the health management function to be provided, on a basis of an indicator included in the user information; and
setting a mode of providing the health management function according to a result of the determination, wherein
the information processing terminal includes an audio output unit,
the health management function includes presenting information via the audio output unit,
the indicator includes an indicator relating to hearing acuity of the user, and
the setting of a mode of providing the health management function includes changing at least one of volume of audio, pitch of audio, and playback speed of audio output by the audio output unit in accordance with the suitability determined on a basis of an indicator relating to the hearing acuity.

14. A health management function providing method using an information processing terminal configured to execute an application recording medium for providing a health management function relating to management of health of a user, the method comprising:
acquiring user information including an indicator relating to health of the user;
performing a determination of suitability of the user for the health management function to be provided, on a basis of an indicator included in the user information; and
setting a mode of providing the health management function according to a result of the determination, wherein
the health management function includes presenting to the user a method of performing exercise relating to treatment of a disease or injury of the user,
the indicator includes an indicator relating to at least one of body temperature, blood pressure, pulse, and electrocardiogram of the user, and
the setting of a mode of providing the health management function includes presenting information indicating that the exercise should be stopped in accordance with the suitability.

15. A recording medium for causing an information processing apparatus to execute each of steps of the health management function providing method according to claim 11.

16. A recording medium for causing an information processing apparatus to execute each of steps of the health management function providing method according to claim 12.

17. A recording medium for causing an information processing apparatus to execute each of steps of the health management function providing method according to claim 13.

18. A recording medium for causing an information processing apparatus to execute each of steps of the health management function providing method according to claim 14.
